(19) United States
(12) Patent Application Publication
(10) Pub. No.: US 2011/0059094 A1
(43) Pub. Date: Mar. 10, 2011

(54) ANTITUMOR EFFECTS OF INSULIN-RESPONSIVE DNA BINDING PROTEIN-1 (IRDBP-1)

(76) Inventor: Betty C. VILLAFUERTE, Atlanta, GA (US)

(21) Appl. No.: 12/876,484

(22) Filed: Sep. 7, 2010

Related U.S. Application Data
(60) Provisional application No. 61/240,643, filed on Sep. 8, 2009.

(51) Int. Cl. A61K 39/395; C12N 5/09; A61K 31/711; C07K 16/18; C12Q 1/68; A61P 35/00; C12N 15/85

(52) U.S. Cl. 424/139.1; 435/375; 514/44 R; 530/387.9; 435/6; 435/455

(57) ABSTRACT

Methods of inhibiting growth of tumor cells with a nucleic acid encoding IRDBP-1 or the carboxyl segment thereof are disclosed. Also disclosed are methods for treating tumor with an antibody specific against one of the epitopes in the amino segment of IRDBP-1. In addition, methods of identifying a compound that affects the activity of IRDBP-1 in a cell are disclosed.

### REFERENCE TO RELATED APPLICATION

The present application claims the priority to U.S. Provisional Application Ser. No. 61240643, filed Sep. 8, 2009, which is herein incorporated by reference in its entirety.

### ACKNOWLEDGMENT OF FEDERAL RESEARCH SUPPORT

This invention was made, at least in part, with funding from the National Institutes of Health (DK067413). Accordingly, the United States Government has certain rights in this invention.

### FIELD OF THE INVENTION

The present invention relates generally to DNA-binding proteins, and more specifically to insulin-responsive DNA Binding Protein-1 (IRDBP-1).

### BACKGROUND OF THE INVENTION

Obesity has emerged as an important risk factor for many types of cancer. The metabolic consequences of obesity, including insulin resistance, appears to underlie the obesity-cancer association. Insulin resistance is a condition in which the responsiveness of target cells to insulin signaling is impaired and is countered by an increased production of insulin. One in three Americans has insulin resistance, and there is accumulating epidemiological evidence linking insulin resistance and increased cancer risk. For example, patients with type 2 diabetes have a higher rate of colon cancer (Hu et al., (1999) “Prospective study of adult onset diabetes mellitus type 2 and risk of colorectal cancer in women” Journal of National Cancer Institute 91: 542-547), and type 2 diabetics with colon cancer have a higher recurrence rate and worse prognosis (Meyerhardt et al., (2003) “Impact of diabetes mellitus on outcomes in patients with colon cancer” Journal of Clinical Oncology 21: 433-440). In addition to an increased risk for colon cancer, recent epidemiological studies reported a link between obesity and the development of other common cancers, including breast, lymphoma, prostrate, kidney, pancreas, and endometrial cancers (Bordeux et al, (2006) “Beyond cardiovascular risk: The impact of obesity on cancer death, Cleveland Clinic Journal of Medicine”73: 945-950).

There is a need to understand the mechanism by which insulin influences tumor promotion and growth and to identify new targets for development of anti-cancer agents.

### SUMMARY OF THE INVENTION

In one aspect, the invention relates to a method of inhibiting growth of tumor cells, comprising: introducing a DNA construct encoding IRDBP-1 protein into the tumor cells; and allowing the DNA construct to express the IRDBP-1 protein in the tumor cells, thereby inhibiting growth of the tumor cells; wherein the IRDBP-1 protein comprises the amino acid sequence selected from the group consisting of SEQ ID NOs: 2 and 5.

In another aspect, the invention relates to a method of inhibiting growth of tumor cells, comprising: introducing a DNA construct comprising the nucleotide sequence selected from the group consisting of SEQ ID NOs: 1 and 6; and allowing the DNA construct to express IRDBP-1 protein in the tumor cells, thereby inhibiting growth of the tumor cells.

In another aspect, the invention relates to a method of inhibiting growth of tumor cells, comprising: increasing nuclear translocation of the carboxyl fragment of IRDBP-1 in the tumor cells, thereby inhibiting growth of the tumor cells; wherein the carboxyl fragment of IRDBP-1 comprises the amino acid sequence of SEQ ID NO: 5.

In another aspect, the invention relates to an antibody specifically against one of the epitopes chosen from the amino acid sequence selected from the group consisting of SEQ ID NOs: 2 and 10.

In another aspect, the invention relates to a method of inhibiting growth of tumor cells, comprising exposing the tumor cells to an effective amount of the antibody specifically against one of the epitopes chosen from the amino acid sequence of SEQ ID NO: 10, and thereby inhibiting growth of the tumor cells.

In another aspect, the invention relates to a method of inhibiting growth of tumor cells in a patient, comprising exposing the tumor cells in the patient to an effective amount of the antibody specifically against one of the epitopes chosen from the amino acid sequence of SEQ ID NO: 10, and thereby inhibiting growth of tumor cells in the patient.

Further in another aspect, the invention relates to a method for identifying a compound that affects activity of IRDBP-1 in a cell, comprising: providing a cell transfected with a first vector and a second vector, the first vector comprising a reporter gene operably linked to IGFBP-3 promoter insulin responsive element (IRE) and the second vector comprising a DNA insert encoding a polypeptide comprising the amino acid sequence of SEQ ID NO: 5 operably linked to a constitutive promoter; exposing the cell to a test compound; detecting the level of the expression product of the reporter gene expression in the cell exposed to the test compound; and comparing the level of the expression product of the reporter gene expression in the cell exposed to the test compound with the level of the expression product of the reporter gene expression in the cell not exposed to the test compound to detect whether the test compound affects activity of IRDBP-1 in the cell; wherein an increase in the level of the expression product of the reporter gene expression in the presence of the test compound relative to the level of the expression product of the reporter gene expression in the absence of the test compound is an indication of a potential agonist of the test compound.

Yet in another aspect, the invention relates to a method of identifying a compound that affects the activity of IRDBP-1 in a cell comprising:
providing a cell transfected with a nucleic acid insert comprising:
   a) a constitutive promoter;
   b) a cDNA encoding IRDBP-1, the cDNA operably linked to the constitutive promoter and the IRDBP-1 comprising the amino acid sequence of SEQ ID NO: 2; and
   c) a reporter gene operably linked to the cDNA at the 5′ end thereof;
exposing the cell to a test compound;
detecting the expression product of reporter gene expression in the cell exposed to the test compound; and
comparing the intracellular localization of the expression product of the reporter gene expression in the cell exposed to the test compound with the intracellular localization of the expression product of the reporter gene expression in the cell not exposed to the test compound to determine whether the test compound affects the activity of IRDBP-1 in the cell;
wherein an increase in nuclear detection of the expression product of the reporter gene expression is an indication of a potential agonist of the test compound.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1A shows microscopic images of HepG2 human liver carcinoma cells showing cell localization of IRDBP-1 by immunostaining.

FIG. 1B are autoradiographs of western blot showing the molecular sizes and cell compartmentalization of IRDBP-1 protein fragments.

FIG. 2A shows liver sections from a normoglycemic lean rat (left panel) and a hyperglycemic obese diabetic rat (right panel) immunostained with the carboxyl antibody of IRDBP-1.

FIG. 2B is a schematic drawing illustrating the structure of IRDBP-1 as predicted by SMART (Simple Modular Architecture Research Tool, EMBL).

FIG. 3 is a northern blot autoradiograph showing relative expressions of IRDBP-1 mRNA in various parts of the human gastrointestinal tract.

FIG. 4 is a photograph showing dot blot analysis of IRDBP-1 mRNA from tumor and matched normal tissues.

FIG. 5 shows tissue specimens from breast carcinoma and corresponding normal breast tissues of the same human patient immunostained with IRDBP-1 carboxyl segment antibody. Similar stain pattern was detected in n=6.

FIG. 6 shows a human colon carcinoma tissue specimen immunostained with IRDBP-1 carboxyl segment (or fragment) antibody, counterstained with hematoxylin to show nuclear compartments. Left, 10× magnification. Right, 100× magnification. Similar stain pattern was detected in n=28.

FIG. 7 shows human rectal cancer tissue specimen immunostained with IRDBP-1 carboxyl segment antibody. The specimen was further stained with hematoxylin for nuclear staining.

FIG. 8 shows human pancreas immunostained with IRDBP-1 carboxyl segment antibody. The specimen was further stained with hematoxylin for nuclear staining. Left, normal pancreas. Right, pancreatic cancer. Similar pattern of staining was detected in n=5.

FIG. 9 shows human B-cell, T-cell, and Hodgkin's lymphomas immunostained with pre-immune serum or IRDBP-1 carboxyl segment antibody. The specimens were further stained with hematoxylin for nuclear staining.

FIG. 10 is a graph showing the effect of IRDBP-1 expression on soft agar colony formation of human colon cancer cells after 4 weeks. * indicates p<0.05 vs control.

FIG. 11A shows the effect of IRDBP-1 expression on colon cell tumor growth in nude mice. Two mice, each injected with control vector-transfected HT29 colon cells in their left flank, and with IRDBP-1 cDNA vector-transfected HT29 colon cells in their right flank, are shown.

FIG. 11B is a graph showing the effect of IRDBP-1 expression on tumor weight after 28 days of growth in nude mice, n=6/group.

FIGS. 11C-11D show tissue sections of xenographs from nude mice 28 days after injection with control vector-transfected HT29 cells, stained with hematoxylin and eosin.

FIG. 11E shows a tissue section of xenograph from a nude mouse 28 days after injection with IRDBP-1 cDNA vector-transfected HT29 cells, stained with hematoxylin and eosin.

FIG. 12 is a graph showing the effect of siRNA knockdown of IRDBP-1 on cell proliferation.

FIG. 13A shows histology of lymphoma tumors obtained from mice after transfection of EL4 cells with adenovirus expressing IRDBP-1 or control vector only (upper panel). Xenograft sections were immunostained with anti-VEGF antibody, and counterstained with hematoxylin (lower panel).

FIG. 13B is a graph showing lymphoma rumor volume with or without transfection with IRDBP-1.

FIG. 13C is a graph showing survival curves of mice treated with vehicle, empty vector, or IRDBP-1 cDNA.

FIG. 14A shows the effect of IRDBP-1 amino fragment-specific antibody on the translocation of the IRDBP-1 carboxyl fragment in SW480 colon cancer cells.

FIG. 14B shows the expression of the 50 kDa carboxyl fragment of IRDBP-1 18 hrs after treatment of SW480 cells with amino fragment-specific antibody of IRDBP-1 or with control IgG.

FIG. 14C shows the effect of the IRDBP-1 amino fragment-specific antibody on ERK signaling by western blot.

FIG. 15A is a graph showing the effect of the amino fragment-specific antibody of IRDBP-1 on HT29 colon cancer cell proliferation.

FIG. 15B is a graph showing the effect of the amino fragment-specific antibody of IRDBP-1 on SW480 colon cell proliferation.

FIG. 16A shows 3-D models of various peptide fragments of IRDBP-1, as predicted by SWISS-MODEL, generated by homology modeling.

FIG. 16B shows a 3-D model of insulin receptor for comparison.

FIG. 17 is a graph showing the results of screening for IRDBP-1 agonists.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, when a number or a range is recited, ordinary skill in the art understand it intends to encompass an appropriate, reasonable range for the particular field related to the invention.

As used herein, the term “IRDBP-1” refers to an Insulin-Responsive DNA Binding Protein-1 capable of binding to at least one insulin responsive element associated with a gene or genes, and by so doing so may regulate the expression of an insulin-responsive gene. The term “IRDBP-1” is also intended to apply to proteins, peptides or polypeptides capable of binding to at least one insulin-responsive element of eukaryotic organisms, including humans. Without intent to limit the scope of the invention, examples of IRDBP-1 include a polypeptide comprising the amino acid sequence chosen from SEQ ID NO: 2 and SEQ ID NO. 5.

Insulin-response DNA binding protein-1 (IRDBP-1), also known as insulin-response element binding protein-1 (IRE-BP1), is a transcription factor that mediates the metabolic actions of insulin (Villafuerte et al., “Insulin-Response element binding protein 1, a novel Akt substrate involved in transcriptional action of insulin. Journal of Biological Chemistry, 279:36650-36659, 2004). IRDBP-1 is a target of insulin signaling downstream of both the Phosphatidyl inositol 3′ Kinase/Akt and MAP kinase (Erk) pathways. It has been reported that IRDBP-1 decreased insulin resistance and ameliorated hyperglycemia in type 2 diabetes. See U.S. Pat. Nos. 7,563,775 and 7,635,766, which are incorporated herein by reference in their entireties. The present invention relates to the discovery of the utility of IRDBP-1 in controlling tumor growth and cell proliferation.

In one aspect, the invention relates to a method of inhibiting growth of tumor cells, comprising: introducing a DNA construct encoding IRDBP-1 protein into the tumor cells; and allowing the DNA construct to express the IRDBP-1 protein in the tumor cells; thereby inhibiting growth of the tumor cells; wherein the IRDBP-1 protein comprises the amino acid sequence selected from the group consisting of SEQ ID NOs: 2 and 5.

In one embodiment of the invention, the IRDBP-1 protein comprises a amino acid sequence that has at least 95% homology to SEQ ID NO: 2 or SEQ ID NO: 5.

In one embodiment of the invention, the IRDBP-1 protein comprises a amino acid sequence that has at least 90% homology to SEQ ID NO: 2 or SEQ ID NO: 5.

In another aspect, the invention relates to a method of inhibiting growth of tumor cells, comprising: introducing a DNA construct comprising the nucleotide sequence selected from the group consisting of SEQ ID NOs: 1 and 6; and allowing the DNA construct to express IRDBP-1 protein in the tumor cells, thereby inhibiting growth of the tumor cells.

In one embodiment of the invention, the DNA construct comprise an insert comprising a nucleotide sequence that has at least 90% homology to SEQ ID NO: 1 or SEQ ID NO: 6.

In one embodiment of the invention, the DNA construct comprises a nucleotide sequence that has at least 95% homology to SEQ ID NO: 1 or SEQ ID NO: 6.

In another embodiment of the invention, the DNA construct comprises a nucleotide sequence that has at least 90% homology to SEQ ID NO: 1 or SEQ ID NO: 6.

In one embodiment of the invention, the tumor cells are present in a mammal.

In another aspect, the invention relates to a method of inhibiting growth of tumor cells, comprising increasing nuclear translocation of the carboxyl fragment of IRDBP-1 in the tumor cells, thereby inhibiting growth of the tumor cells; wherein the carboxyl fragment of IRDBP-1 comprises the amino acid sequence of SEQ ID NO: 5.

In one embodiment of the invention, the method further comprises exposing the tumor cells to an effective amount of an antibody that is specific against one of epitopes chosen from the amino fragment of IRDBP-1, the amino fragment comprising the amino acid sequence of SEQ ID NO: 10.

In another embodiment of the invention, the tumor cells are selected from the group consisting of colon cancer cells, rectal cancer cells, pancreatic cancer cells, breast cancer cells, kidney cancer cells, lymphoma cells, and any combination thereof.

In another aspect, the invention relates to an antibody specifically against one of the epitopes chosen from the amino acid sequence selected from the group consisting of SEQ ID NOs: 2 and 10.

In another aspect, the invention relates to a method of inhibiting growth of tumor cells, comprising exposing the tumor cells to an effective amount of the antibody specifically against one of the epitopes chosen from the amino acid sequence of SEQ ID NO: 10, and thereby inhibiting growth of the tumor cells.

Alternatively, the method comprises exposing the tumor cells in the patient to an effective amount of the antibody specifically against one of the epitopes chosen from the amino acid sequence of SEQ ID NO: 10, and thereby inhibiting growth of tumor cells in the patient.

In one embodiment of the invention, the antibody is specific against an epitope comprising the amino acid sequence of SEQ ID NO: 3.

Gene therapy using an IRDBP-1 expression vector has been described in U.S. Pat. No. 7,563,775. IRDBP-1 or carboxyl fragment-encoding DNA may be delivered into cells using viral vectors such as recombinant adenovirus, retrovirus, adeno-associated virus, herpes simplex virus-1, recombinant bacterial or eukaryotic plasmids. The IRDBP-1 gene in the DNA construct may operably linked to a constitutive promoter, an inducer, or an enhancer, which are known in the art.

In clinical settings, a gene delivery system may be introduced into a patient by any method known in the art by intravenous injection. Specific transduction of the IRDBP-1 or carboxyl fragment-encoding gene into target cells relies predominantly on the specificity of transfection provided by the gene delivery vehicle, cell-type or tissue-type expression due to the transcriptional regulatory sequences controlling expression of the gene, or a combination thereof.

Initial delivery of recombinant gene may be limited to an introduction into a local tissue of the animal. Delivery of IRDBP-1 or carboxyl fragment-encoding gene into the liver by hepatic artery infusion has been reported in the art. For example, a gene delivery vehicle can be introduced by catheter (see U.S. Pat. No. 5,328,470) or by stereotactic injection (e.g. Chen et al. (1994) Proc. Natl. Acad. Sci. 91: 3054-3057) into the liver, both of which references are incorporated herein in their entireties. Acceptable diluent may be added. Alternatively, a slow release matrix imbedded with the gene delivery vehicle may be used.

Antibodies may be used to bind to IRDBP-1 and inhibit the receptor's binding to ligands and induce nuclear translocation and activation of the carboxyl fragment that results in changes in gene expression. Changes in gene expression results in decrease tumor growth. The antibody may be a full-length antibody, a substantially intact antibody, a Fab fragment, a F(ab′)₂ fragment, or a single chain Fv fragment. Antibodies may be administered by any means known in the art that achieve the generally intended purpose to treat cancer. The preferred route of administration is parenteral. The concentration of anti-IRDBP-1 antibody in the formulation may be from as low as 0.1% to as much as 20% by weight, generally in the range of 1 to 100 mg/mL, or present in an amount that is effective to achieve the desired medical effect of treating cancer.

Further in another aspect, the invention relates to a method for identifying a compound that affects activity of IRDBP-1 in a cell, comprising: providing a cell transfected with a first vector and a second vector, the first vector comprising a reporter gene operably linked to IGFBP-3 promoter insulin responsive element (IRE) and the second vector comprising a DNA insert encoding a polypeptide comprising the amino acid sequence of SEQ ID NO: 5 operably linked to a constitutive promoter; exposing the cell to a test compound; detecting the level of the expression product of the reporter gene expression in the cell exposed to the test compound; and comparing the level of the expression product of the reporter gene expression in the cell exposed to the test compound with the level of the expression product of the reporter gene expression in the cell not exposed to the test compound to detect whether the test compound affects activity of IRDBP-1 in the cell; wherein an increase in the level of the expression product of the reporter gene expression in the presence of the test compound relative to the level of the expression product of the reporter gene expression in the absence of the test compound is an indication of a potential agonist of the test compound.

In one embodiment of the invention, the reporter gene is one chosen from firefly luciferase and β-galactosidase, and green fluorescent protein.

In another embodiment of the invention, the constitutive promoter is a CMV promoter. Yet in another aspect, the invention relates to a method of identifying a compound that affects the activity of IRDBP-1 in a cell comprising:
providing a cell transfected with a nucleic acid insert comprising:
   a) a constitutive promoter;
   b) a cDNA encoding IRDBP-1, the cDNA operably linked to the constitutive promoter and the IRDBP-1 comprising the amino acid sequence of SEQ ID NO: 2; and
   c) a reporter gene operably linked to the cDNA at the 3′ end thereof;
exposing the cell to a test compound;
detecting the expression product of reporter gene expression in the cell exposed to the test compound; and
comparing the intracellular localization of the expression product of the reporter gene expression in the cell exposed to the test compound with the intracellular localization of the expression product of the reporter gene expression in the cell not exposed to the test compound to determine whether the test compound affects the activity of IRDBP-1 in the cell;
wherein an increase in nuclear detection of the expression product of the reporter gene expression is an indication of a potential agonist of the test compound.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### Examples

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Methods

Screening of Human cDNA Libraries for IRDBP-1 Nucleic Acids

To obtain an isolated nucleic acid encoding a region of the human IRDBP-1, a 3401 by rat cDNA (GenBank Accession Number AF439719.1; also see SEQ ID NO: 14 as described in U.S. Pat. No. 7,563,775, which is incorporated herein by reference in its entirety) was used to screen λ phage human brain and liver cDNA libraries (Uni-Zap XR, Stratagene, La Jolla, Calif.) to obtain isolated human nucleic acids capable of hybridizing with the rat cDNA, per Manufacturer's protocol. After isolation of the plaques that hybridized with the 3401 bp rat cDNA probe, the pBluescript phagemid was rescued with the VCSM 12 helper phage, and the human cDNA clones were sent for automatic sequencing. Two human cDNA clones were obtained. One was about 2480 bp long and another clone was about 1700 bp long. Subsequent 5′ extension were performed by 5′RACE techniques, using the SMART RACE cDNA amplification system from Clontech, Inc. and polyA mRNA isolated from human cerebellum, until the 6331 bp sequence (SEQ ID. NO: 1) was obtained. BLASTN algorithm searching of the GenBank database using the human IRDBP-1 nucleic acid sequence SEQ ID. NO: 1 as the search target found that there was almost 100% identity with regions of the human genomic DNA sequence GenBank Accession No. XM_{—}059482 from the human chromosome 2q37.3, and a human gene encoding the IRDBP-1 transcribed from nucleic acids that comprised 33 exons. The N-terminus of the IRDBP-1 of SEQ ID. NO: 2 extends 175 amino acids beyond a genomic sequence annotated by the Human Genome Project as SNED1 (Sushi, nidogen and EGF-like domains 1).

### Northern Blot

A multiple tissue northern blot from Clontech Laboratories, Inc (Palo Alto, Calif.) containing 2 μg of poly-adenylated RNA per lane isolated from various human tissues was probed with a radioisotope-labeled human IRDBP-1 cDNA probe. The probe was derived from a 2504 bp cDNA and comprise the nucleotides 3827 to 6331 of SEQ ID NO. 1. Appropriate stringency conditions which promote DNA hybridization in 6X SSC buffer at about 45EC, followed by a wash of 2X SSC at 50EC, were performed. The nylon membrane was then exposed to autoradiographic film and developed. Similar hybridization condition was used to investigate the expression of IRDBP-1 mRNA in matched tumor/normal expression array. Total RNA was obtained from various normal human and tumor tissues to synthesize cDNA by PCR. The PCR amplified cDNA were then blotted onto nylon membrane, which was then hybridized with the radioisotope-labeled human IRDBP-1 cDNA probe as described above.

### IRDBP-1 Specific Antibodies

Antibodies were developed against oligopeptides corresponding to regions in the amino and carboxyl segments of IRDBP-1. The antibody specific against the amino segment of IRDBP-1 was raised against an epitope of human IRDBP-1 protein located between the amino acids Thr105-Lys121 of SEQ ID NO: 2. The amino terminal antibody sequence is: Acetylated Thr-Arg-Ala-Pro-Arg-Arg-Ala-Ser-Gly-Ser-Arg-Gln-Ala-Leu-Asp-Arg-Lys-amide (SEQ ID NO: 3). A carboxyl segment-specific antibody was generated against an epitope at the carboxyl segment of IRDBP-1 at the amino acids Ser1247-Arg1264, having the following sequence: Acetylated Ser-Pro-Arg-Asp-Gly-Ala-Asp-Arg-Arg-Trp-His-Gln-Gly-Gly-His-His-Pro-Arg-amide (SEQ ID NO: 4).

### Immunodetection of IRDBP-1 Expression in Breast, Colon, Rectal, Pancreatic, Lymphatic Cancers, and in Hepatocytes

Fixed and paraffin embedded surgical tissue specimens obtained from humans with various cancers were deparaffinized, rehydrated, treated with proteinase K at 50 microgram/ml for 10 min at room temperature, washed with PBS, and blocked with 5% BSA/1% goat serum/PBS mixture for 1 hr. The primary antibody anti-human IRDBP-1 carboxyl segment antibody was added at 1:200 dilution in 1% BSA/1×PBS, and the sample was incubated in a humid chamber overnight at 4° C. After washing, a biotinylated secondary antibody was added at 1:400 dilution, and incubated with the sample for 60 mins. Color development was performed with DAB complex from an alkaline phosphatase standard kit. The slide was counterstained with Gill's hematoxylin, dehydrated, and mounted. The level of IRDBP-1 carboxyl segment was measured in tissues produced by biopsy or from surgical specimens.

Liver sections were obtained from diabetic and non-diabetic Zucker rats, fixed with 4% paraformaldehyde, and then blocked with 5% normal goat serum in 1% (wt/vol) BSA-Tris-buffered saline (TBS) at room temperature for 1 hour. This was followed by washing in TBS and overnight incubation with rabbit anti-IRDBP-1 carboxyl segment antibody at 1:150 dilution in TBS-0.5% Triton X-100. The primary antibody incubation was followed by washing and incubation with Alexa Fluor 488 goat antirabbit IgG (Invitrogen) at 1:500 dilution. The cells were embedded in mounting medium, and optical sections in the center of the nuclei were obtained with a Zeiss Axiovert 100M confocal microscope.

### Adenovirus Vector Expressing IRDBP-1 Carboxyl Fragment

Using the AdEasy system (Qbiogene, Inc.), the 1380 bp fragment of human IRDBP-1 cDNA that comprised the carboxyl fragment of the protein-coding sequence was cloned into a shuttle vector (pShuttle-CMV). Once constructed, the shuttle vector was linearized with Pme1 and cotransformed into BJ5183 together with AdEasy-1, the supercoiled viral DNA plasmid. Transformants were selected for kanamycin resistance, and recombinants were subsequently identified by restriction digestion. Purified recombinant Ad Plasmid DNA was digested with Pad to expose its inverted terminal repeats and then used to transfect AD-293 cells where deleted viral assembly genes were complemented in vivo. Then recombinant adenovirus was obtained by plaque purification, amplified, and purified. The adenovirus expressing IRDBP-1 carboxyl fragment was introduced into EL4 lymphoma cells by transfection and the transfected cells injected subcutaneously into C6 black mice, and the effects of IRDBP-1 expression on tumor growth and histology and the survival of the mice were investigated.

### Stable Transfection of IRDBP-1 Carboxyl Fragment in HT29 Colon Cancer Cells

A 1380-bp carboxyl fragment of human IRDBP-1 cDNA (SEQ ID NO: 6, i.e., the nucleotide 3510 to nucleotide 4889 of SEQ ID NO. 1 (MW of 50,709.31)), encoding the carboxyl fragment of IRDBP-1 (SEQ ID NO: 5) was subcloned into the pCMV-tag2 vector (Stratagene, La Jolla, Calif.) at the SmaI site. Stable transfections were achieved with Targefect F2 and Virofect enhancer (Targeting Systems, San Diego, Calif.). Human HT29 colon cancer cells grown in 60 mm dishes at 60% confluence were transfected with 6 μg of the IRDBP-1/pCMV-Tag2 construct or a control vector (without the insert) overnight, followed by 48 h recovery in fresh media. After the recovery period, G418 was added at 800 μg/ml for 21 days. The colonies were picked and expanded under G418 selection. Cells with stable expression of IRDBP-1 carboxyl fragment or transfected with the control vector were used in the studies of the cell proliferation effects on soft agar colony formation. The stably transfected cell lines were also injected into the flanks of female nude mice, and the tumor growth assessed after 28 days.

Small Interfering RNA (siRNA) Synthesis and Transfection

Target selection for silencing of IRDBP-1 was designed according to the siRNA guidenlines published in Technical Bulletin # 506 by Applied Biosystems, and the siRNA synthesized according to the manufacturer's protocol (Silencer siRNA Construction Kit from Ambion, Austin, Tex.). Two 29-oligomer DNA oligonucleotides comprising 21-nucleotide sequences corresponding to the sense and antisense strand of IRDBP-1 at nucleotide 2151 to nucleotide 2169 of SEQ ID NO: 1 (Sense: 5′-AAGCATGGCTGTGATTTCAAACCTGTCTC-3′; SEQ ID NO: 7; Antisense: 5′-AATTTGAAATCACAGCCATGCCCTGTCTC-3′; SEQ ID NO: 8) were synthesized with an eight-nucleotide leader sequence complementary to the T7 promoter primer sequence (5′-CCTGTCTC-3′) attached at the 3′ ends for both strands. The DNA regions were selected based on the lack of homology to the coding sequence of other genes. The oligonucleotides were hybridized to T7 promoter primer, extended by klenow DNA polymerase to create double-stranded templates, and then transcribed by T7 RNA polymerase to produce double-stranded RNA. The T7 primer sequences were removed by digestion with ribonuclease H, and the end product was a double-stranded 21-oligomer siRNA, with 3′-terminal uridine dimers that target IRDBP-1 mRNA. The siRNA was transfected into FHC normal colon epithelium, HT29, or HCT 116 colon carcinoma cells by electroporation, and MTT assay was conducted to determine the viability of the cells after 24 hours. The siRNA that expressed scrambled sequence (Ambion, Austin, Tex.) was used as negative control.

### Protein Structure Modeling

Using the automated SWISS-MODEL program, the secondary structure of the IRDBP-1 protein molecule was elucidated. The program used a “comparative” or “homology” approach, in which experimentally elucidated structures of related protein family members were used as templates to model the structure of a protein of interest, such as IRDBP-1.

### DNA Plasmid Construction for Screening Assays

The insulin response element (IRE) of the insulin-like growth factor binding protein-3 (IGFBP-3) gene was identified by deletion mapping of the rat IGFBP-3 promoter region, and by DNase 1 footprinting and mutational analysis (Villafuerte et al., (1997) “Identification of an insulin-responsive element in the rat insulin-like growth factor-binding protein-3 gene” Journal of Biological Chemistry 272; 5024-5030), which is incorporated herein by reference in its entirety), and mapped to the −1150 to −1124 base pair region. To obtain the IGFBP-3 IRE concatemer, double-stranded oligonucleotides corresponding to the IGFBP-3 IRE primer (−1150 5′-AATTCAAGGGTATCCAGGAAAGTCTCCTTCTAAG-3′-1117; SEQ ID NO: 9) were annealed, treated with T4 DNA ligase at 16° C. for 10 min, and then gel-purified and ligated upstream of a luciferase gene reporter containing SV 40 promoter (pGL3-promoter from Promega). Orientation of the sequences and the number of tandem repeats were confirmed by dideoxy sequencing, and the plasmid that contained three tandem repeats of the IRE served as the IGFBP-3 IRE luciferase reporter gene.

IRDBP-1 cDNAs were subcloned into pcDNA3. 1 mammalian expression vector (Invitrogen, Carlsbad, Calif.) to express either 1.4 kb, 2.9 kb, or 3.1 kb of the IRDBP-1 cDNA from the carboxyl end, corresponding to the translated protein sequences of 50 kDa (Arg 1004 to Ser1463 of SEQ ID NO. 2, also known as SEQ ID NO. 5), 96 kDa (Cys 581 to Ser 1463 of SEQ ID NO. 2), and 113 kDa (Gly 412 to Ser 1463 of SEQ ID NO. 2), respectively. Then the cDNAs were co-transfected with the IGFBP-3 IRE luciferase reporter into COS-7 cells by lipofection, and the luciferase activity measured after 48 hours. The results were normalized to Renilla luciferase control vector readings.

### Results and Discussion:

### IRDBP-1 as a Transmembrane Protein Regulated by Proteolysis and Nuclear Translocation

Human IRDBP-1 cDNA (SEQ ID NO: 1) encodes 1463 amino acids, a protein of 156 kDa (SEQ ID NO: 2). The coding region of SEQ ID NO: 1, which translates into the protein (SEQ ID NO: 2), comprises the nucleotides 501-4889. The nucleotide 1-500 is the 5′ untranslated sequence, while nucleotides 4890 to 6331 is the 3′ untranslated sequence.

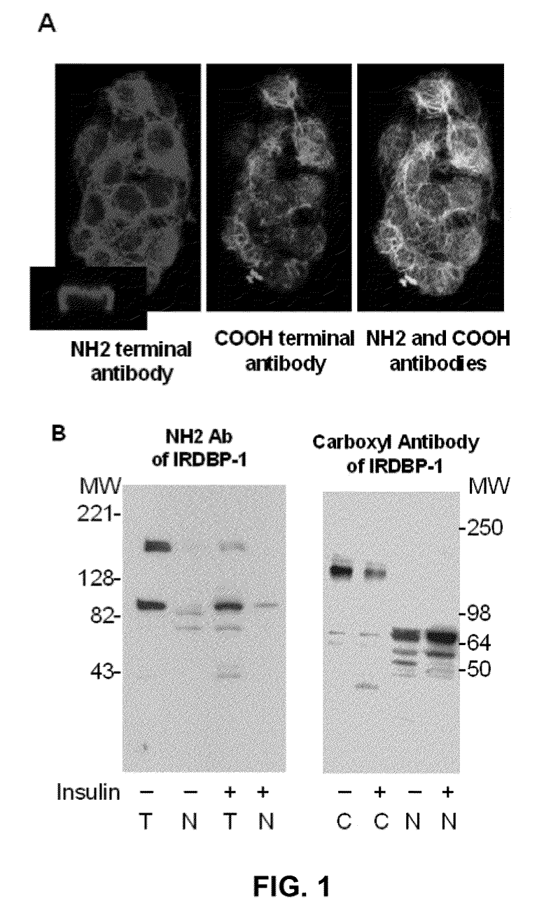

Scanning by hydrophobicity plot showed two hydrophilic and one hydrophobic domains. The hydrophobic region spans a 20 amino acid region between amino acid 531 to 551(residues Leu531-Phe551 of SEQ ID NO: 2), and is predictive of a transmembrane domain by multiple algorithms. To confirm that IRDBP-1 is a transmembrane protein, rabbit polyclonal antibodies against the amino acids Thr105-Lys121 (N-terminal Ab: Ac-TRAPRRASGSRQALDRK-amide; N-terminal epitope; SEQ ID No: 3) and amino acids Ser1247-Arg1264 (C-terminal Ab: Ac-SPRDGADRRWHQGGHHPR-amide; C-terminal epitope: SEQ ID No: 4) of IRDBP-1 were developed to map the protein in the cell. Epitope mapping with confocal microscopy showed that the N-terminal fragment is localized to the outer membrane in non-permeabilized HepG2 cells, and the Carboxyl fragment is mostly nuclear (FIG. 1A). Fractionation of the cellular proteins by sucrose gradient centrifugation showed that a 156- and a 100-kDa protein were detected by the Ab against the N-terminal fragment in the total extract, but this antibody did not detect the N-terminal fragment of the protein in the nucleus, consistent with the finding that the N-terminal fragment is extra-nuclear (FIG. 1B). A 50 kDa fragment was detected in the nucleus by the antibody against the C-terminal fragment, and treatment of cells with insulin facilitates cleavage of the protein, with a ˜100 kDa Amino fragment and a ˜50 kDa Carboxyl fragment predominating. Based on epitope mapping and cell fractionation, evidence supports that IRDBP-1 is a transmembrane protein; the NH2-fragment of the protein is localized outside of the plasma membrane or comprises the ectodomain, while the 50 kDa carboxyl fragment is released from the 156 kDa holoprotein and translocates to the nucleus to activate gene transcription.

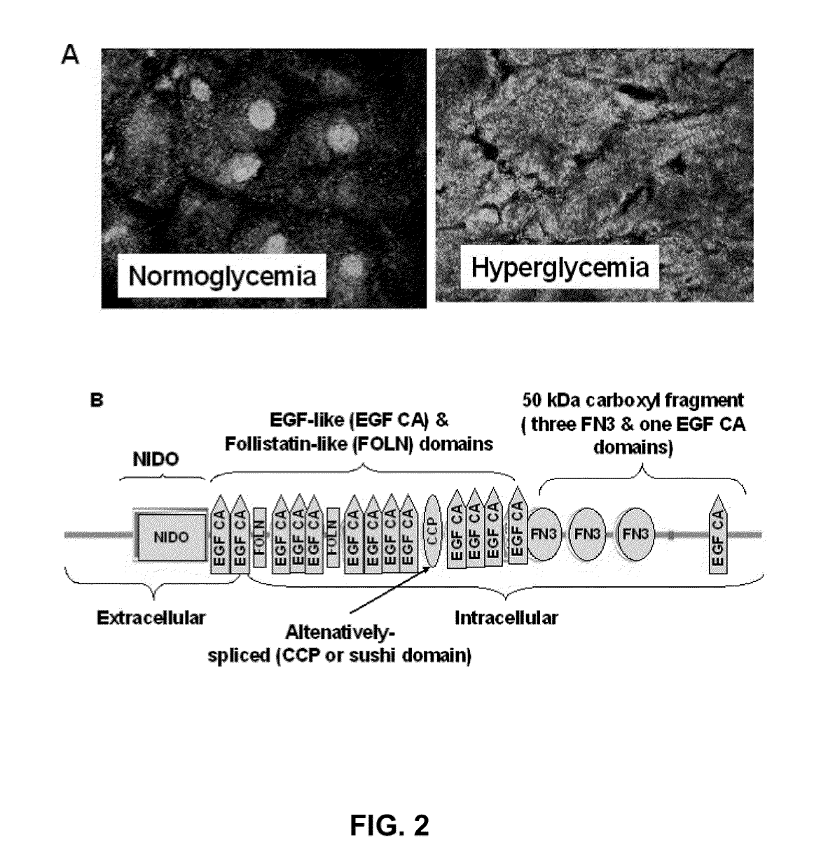

Since IRDBP-1 mediates transcriptional activation of insulin-responsive genes, and the 50 kDa carboxyl fragment cleaved from the 156 kDa holoprotein was detected in the nucleus, the subcellular distribution of the carboxyl fragment in vivo was further investigated. Liver sections were obtained from normal and diabetic rats, and the carboxyl fragment was found to localize predominantly in the nucleus of the cells in normoglycemic lean rats, but was diffusely distributed in both the cytoplasm and the nucleus of the cells in hyperglycemic type 2 diabetic rat liver (FIG. 2A), suggesting that IRDBP-1 was sequestered from its target genes in the nucleus in diabetic animals. Similar cytoplasmic sequestration of carboxyl fragment of IRDBP-1 was detected in the tissue sections of human colon cancer, rectal cancer, and pancreas cancer (FIGS. 6-8). Based on cell fractionation, epitope mapping, and physiological correlation, it was concluded that IRDBP-1 is a single-pass transmembrane protein. The amino (—NH2) fragment comprises the extracellular domain, while the carboxyl fragment comprises the intracellular domain. The biological action of IRDBP-1 requires proteolysis. The 50 kDa carboxyl fragment is cleaved from the 156 kDa holoprotein and translocates to the nucleus to stimulate transcription of insulin-regulated genes (FIG. 2B). In human colon tumors and other tumors, the 50 kDa carboxyl fragment of IRDBP-1 is localized to the cytoplasm rather than the nucleus. This sequesters IRDBP-1 from its target genes in the nucleus of cancer cells and prevents transcription of the genes that have functions in inhibiting tumor cell proliferation or causing tumor regression, or both.

The IRDBP-1 protein contains multiple cysteine-rich motifs. The NH2-domain is predicted to form 13 calcium ion binding epidermal growth factor (EGF_CA)-like repeats (FIG. 2B). Each EGF domain contains −45 amino acids that has six cysteine residues characteristically paired to form disulfide bonds. A sushi-domain (CCP) and two follistatin-like (FOLN) motifs are located within the region comprising the 13 EGF-like repeats (FIG. 2B). The carboxyl-segment of IRDBP-1 is organized into three fibronectin type III (FN3) domains, each of which is a motif containing about 100 amino acids and is structurally similar to that of insulin and IGF-1 receptors. The EGF-like segment of IRDBP-1 has strong similarity to Notch-related proteins, and exhibits 62% homology with the extracellular domain of human Notch receptor 4 (Villafuerte et al. (2004) “Insulin-response Element-binding Protein-1, Journal of Biological Chemistry” 279: 36650-36659). Three consecutive FN3 domains at the carboxyl fragment is similar to the juxtamembrane region of the IGF-1 and insulin receptors (Krammer et al., (1999) “Forced unfolding of the fibronectin type III module reveals a tensile molecular recognition switch” PNAS 94: 1351-56). Based on the existence of structure and sequence similarity to several known cell receptors, it was postulated that a plausible function of IRDBP-1 is to act as a cell receptor. Similar to the Notch receptor, the carboxyl fragment of IRDBP-1 is proteolyzed and released from the membrane-attached holoprotein to act as transcriptional activator.

### IRDBP-1 Inhibits Cancer Formation and Growth

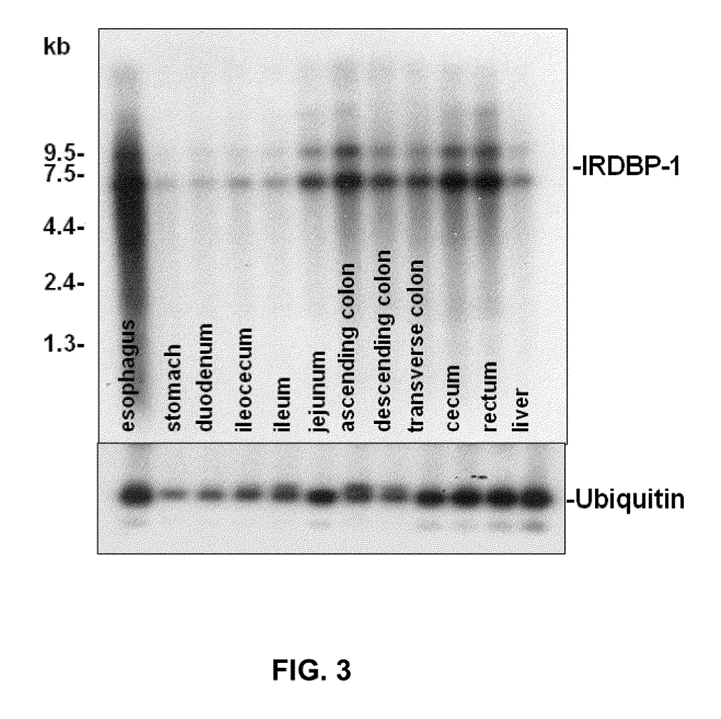

IRDBP-1 may be used for the treatment of various cancers and complications associated with cancers. While conducting studies on the metabolic actions of IRDBP-1, it was discovered that over-expression of IRDBP-1 decrease cellular proliferation. To assess its physiological relevance in humans, tissue distribution of IRDBP-1 in the gastrointestinal tract was examined. A nylon membrane blot (Clontech, Palo Alto, Calif.), in which 2 micrograms of poly A⁺RNA from various parts of the human intestine was blotted, was hybridized with a ³²P-labeled human IRDBP-1 cDNA probe (2504 bp, the nucleotides 3827 to 6331 of SEQ ID NO: 1). Northern blotting showed that IRDBP-1 mRNA is highly expressed in the cells of colon as compared to the cells in most segments of small intestine in normal humans (FIG. 3). The IRDBP-1 mRNA was expressed as two transcripts of about 9 kb and 6 kb. This represents an alternatively-spliced variant (See U.S. Pat. No. 7,563,775).

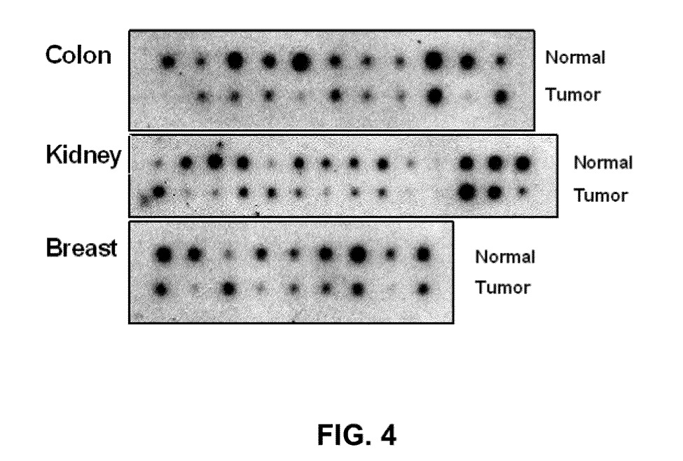

To assess the regulation of IRDBP-1 in human cancers, matched tumor/normal expression array was performed. Briefly, cDNA synthesized from colon, kidney, and breast tumor specimens and corresponding normal tissue from the same human patients were respectively immobilized in separate dots and then hybridized with ³²P-labeled IRDBP-1 human cDNA probe. FIG. 4 shows relative expressions of IRDBP-1 mRNA in various tumors and corresponding normal human tissues. The studies of matched tumor/normal tissue showed that the level of IRDBP-1 mRNA tended to be lower in tumor tissues such as colon tumors, breast tumors, and kidney tumors, as compared to the normal colon, breast, and kidney tissues (FIG. 4).

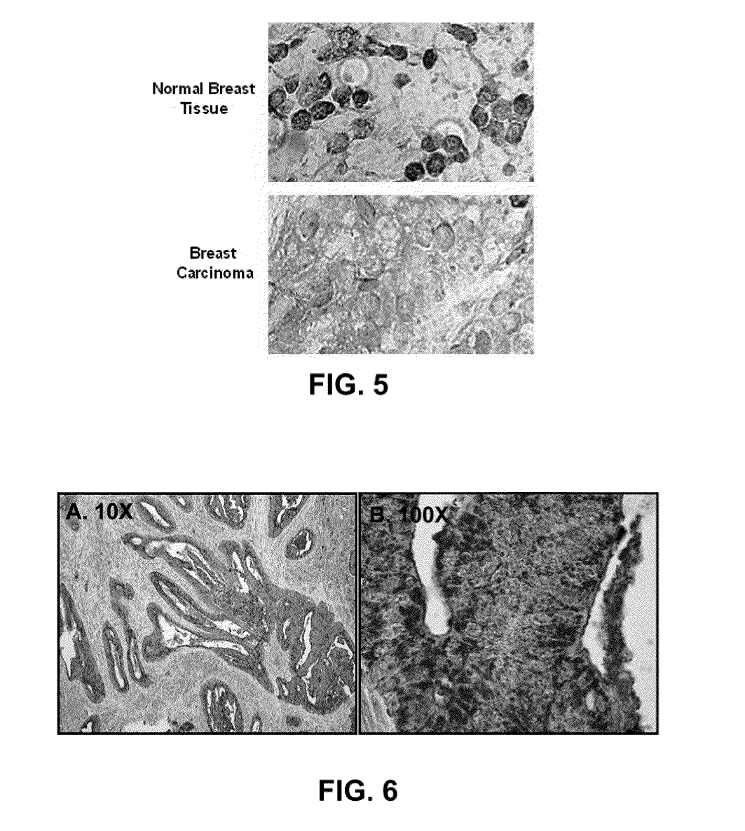

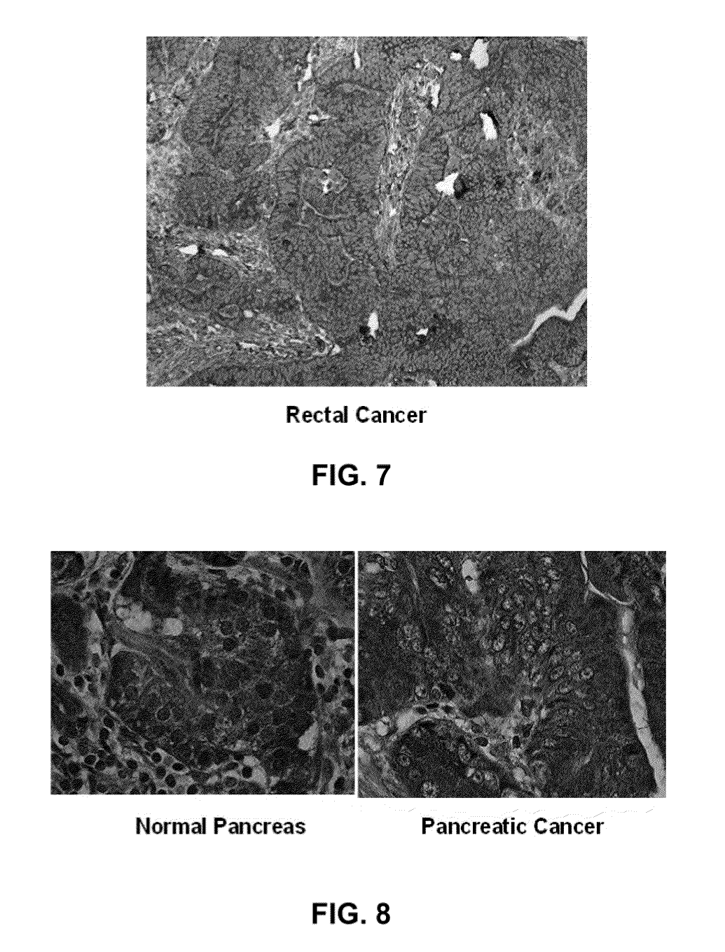

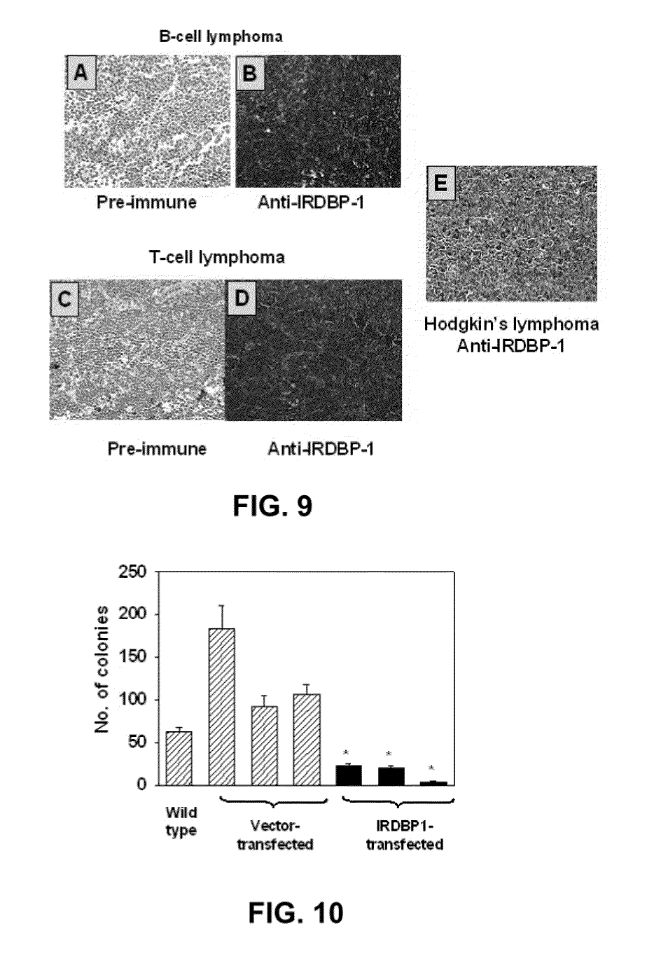

The relevance of IRDBP-1 expression in colon cancer and breast cancer was examined further by investigating the protein expression level and cellular localization in surgical specimens. Immunostaining of surgical specimens with IRDBP-1 C-terminal fragment-specific antibody against the C-terminal epitope (SEQ ID NO: 4) showed a decreased expression of IRDBP-1 protein in six different breast cancer tissues as compared to matched normal breast tissues (FIG. 5). In colon carcinoma specimens, the transcriptionally active 50 kDa carboxyl fragment of IRDBP-1 was localized mainly to the cytoplasm or was excluded from the nucleus, consistent with the finding of impaired nuclear translocation of the protein in colon cancer cells (FIG. 6). Similar sequestration of IRDBP-1 was detected in human rectal carcinoma (FIG. 7) and pancreatic carcinoma specimens (FIG. 8), but diffuse distribution was detected in different types of human lymphomas specimens (FIG. 9). Because IRDBP-1 is excluded from the nucleus in some cancers where it is needed to induce its genomic action, the effects of over-expression of IRDBP-1 in tumor cells was tested in vitro and in different animal models.

To test the effects of IRDBP-1 on colon cancer cell growth in vitro, human colon carcinoma cells (HT29) was transfected with either Flag-tagged pCMV-tag2 vector (Stratagene, La Jolla, Calif.) or pCMV-tag2 expressing 50 kDa carboxyl fragment of IRDBP-1 (pCMV-tag2-IRDBP-1) The cDNA (SEQ ID NO: 6) comprises nucleotide 3510 to nucleotide 4889 of SEQ ID NO. 1, which encodes the amino acids of SEQ ID NO: 5 (Arg1004 to Ser1463 of SEQ ID NO: 2). Stable cell lines were established by G418 antibiotic selection. After seeding stably-transfected cells, the number of colonies that grew on soft agar to larger than 5 mm were compared after 4 weeks (FIG. 10). Compared to the parental cell line, the number of colonies formed by three separate vector-transfected cell lines increased by 1.5-2.9 fold (p<0.05 vs control), and the colonies formed by three IRDBP-1 carboxyl fragment-transfected cell lines decreased by 65±5, 67±4, and 94±1% (all p<0.05 vs control). Thus, expression of IRDBP-1 carboxyl fragment decreased anchorage-dependent growth of HT29 cells in soft agar colony assay. Black bars represent three different cell lines of HT29 human colon cancer cells stably transfected with a vector expressing the 50 kDa carboxyl fragment of IRDBP-1. Hatched bars represent three different cell lines transfected with empty vector (without the carboxyl fragment of IRDBP-1). Wild type represent HT29 cells without DNA transfected. HT29 cells were seeded at 1,000 cells/60 mm plate and grown in soft agar. The cells were stained with methylene blue after 4 weeks, and the colonies counted. * indicates p<0.05 vs control.

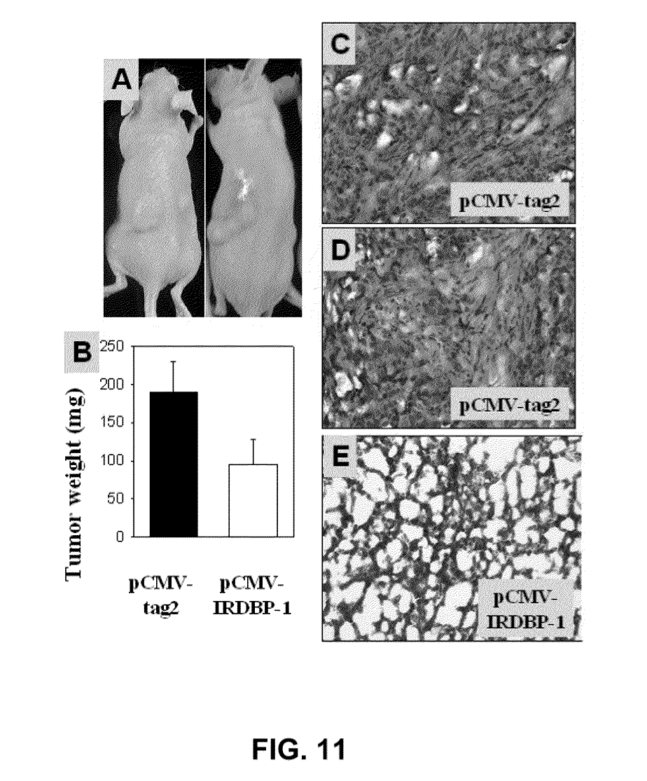

To test the effect of IRDBP-1 on tumor growth in vivo, the above transfected HT29 stable cell lines were injected into the flanks of female nude mice. After 28 days, the mean tumor weight was 190±40 mg (n=6) in the vector-transfected cell lines, whereas tumor weight was significantly lower in each of the IRDBP-1 carboxyl fragment-transfected tumors (95±35 mg, p<0.05 vs control). FIG. 11 illustrates the effect of IRDBP-1 expression on human colon cancer growth in nude mice. Left panel: Six individual HT29 cell lines with stable expression of empty vector (black bar) and 6 cell lines with stable expression of the 50 kDa carboxyl fragment of IRDBP-1 (white bar) were injected into the left flank (vector) or right flank (IRDBP-1) of nude mouse at 2×10⁶ cells/each. The tumors were removed and weighed after 28 days.

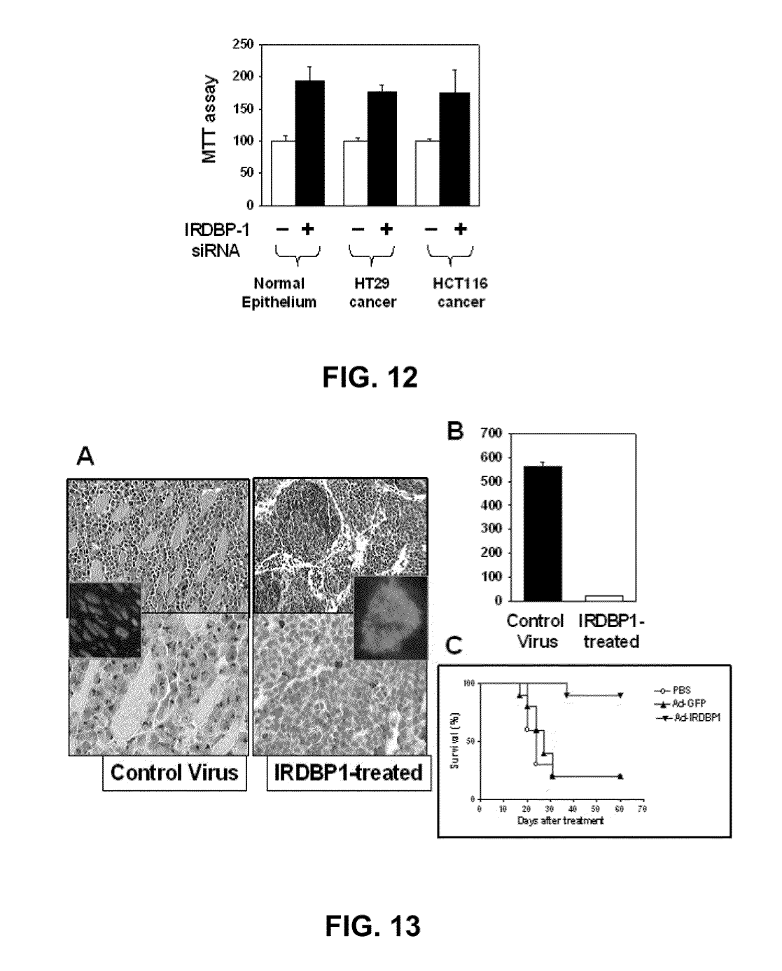

Therefore, constitutively stable expression of IRDBP-1 carboxyl fragment in colon cancer cells inhibited tumor growth in nude mice. Histological studies showed tumors in vector-transfected HT29 cells exhibited features characteristics of epithelial tumors, with pleomorphism and increased microvessel density, forming nested cells separated by fibroblasts (FIGS. 11C and 11D). By contrast, IRDBP-1 carboxyl fragment-transfected cancer cells developed significantly smaller tumors with a cribriform-like histological pattern, with a complete absence of blood vessels (FIG. 11C), suggesting that IRDBP-1 inhibited tumor growth through a decrease in vascularization and fibroblasts formation. Using siRNA strategy (FIG. 12), it was found that knockdown of endogenous IRDBP-1 enhanced cell viability in both normal and transformed colon epithelial cells, including HT29 and HCT116 colon carcinoma cells, as assessed by MTT assay. FIG. 12 shows the effect of siRNA knockdown of IRDBP-1 on cell proliferation. Normal colon and human colon carcinoma cells (HT29 and HCT116) were transfected with scrambled sequence (white bar) or siRNA targeted against IRDBP-1 by electroporation, and cell proliferation assessed by MTT assay 24 hr after transfection. N=8/group.

To determine whether the tumor-inhibitory effect of IRDBP-1 was applicable to other cancer cell types, the effect of IRDBP-1 on lymphoma tumor growth was tested. EL4 lymphoma cells (2×10⁶ cells) were mixed with either adenovirus expressing green fluorescent protein (Ad GFP) or adenovirus expressing a chimeric protein of IRDBP-1 conjugated to GFP (Ad-IRDBP-1) at 2×10⁸ plaque forming units, and injected into the each flank of the C6 black mouse. As shown in FIG. 13, adenoviral delivery of IRDBP-1 into the EL4 lymphoma cells transformed the high-grade malignant lymphoma tumors that infiltrated skeletal muscles (seen as eosinophilic bundles) into histologically benign lymph nodules, observed as formation of germinal centers (FIG. 13A). Gene therapy with IRDBP-1 decreased also the size of the tumors significantly (563 mm³ with control virus vs 21 mm³ with IRDBP-1 treatment, p<0.001, FIG. 13B). Furthermore, IRDBP-1 expression substantially increased the survival rate of the mice (FIG. 13C), and decreased the expression of vascular endothelial growth factor (VEGF), a marker for angiogenesis and cancer metastasis (FIG. 13A, lower panel). Compared to the pleomorphic characteristics of malignant cells, IRDBP-1 expression transformed EL4 cells into smaller and more uniformly-sized cells. Thus, IRDBP-1 acted through cell differentiation and anti-angiogenesis to decrease tumor growth, and its interventions to increase IRDBP-1-mediated actions in the nucleus may be used as a target for treating tumors of multiple cell types.

FIG. 13 shows the effect of IRDBP-1 transfection on EL4 lymphoma tumor growth. A (upper panel): H & E stained tumors obtained from C6 black mice 18 days after injection into each flank of EL4 lymphoma cells transfected with control adenovirus (Ad GFP) or adenovirus expressing IRDBP-1 (Ad IRDBP-1). A (lower panel): Xenografts from EL4 cells treated with control virus or Ad IRDBP-1 were immunostained with anti-human VEGF (vascular endothelial growth factor) antibody, and counterstained with hematoxylin. Insets showed detection of GFP and IRDBP-1-GFP chimeric proteins in the tumors with immunofluorescent microscope. B. Tumor volume 28 days after injection of EL4 lymphoma cells transfected with Ad GFP (black bar) or Ad IRDBP-1 (white bar), n=42/group, P<0.001. C. Survival curves of mice bearing tumor cells transfected with vehicle only (PBS), control virus, or Ad IRDBP-1 N=14/group. P<0.001 for IRDBP-1 vs. PBS & control virus.

### IRDBP-1 as an Immunogenic Target for Cancer Therapy

Since the 100 kDa NH2 fragment of IRDBP-1 remains anchored to the plasma membrane, the experiments were carried out to determine whether antibody neutralization of IRDBP-1 on the cell surface affected the mitogenic signaling of serum growth factors. Colon carcinoma cells were treated with the NH₂-segment antibody of IRDBP-1 (against the N-terminal epitope SEQ ID NO: 3), and tested the effect of the antibody on the activation of the MAP kinase-Erk pathway.

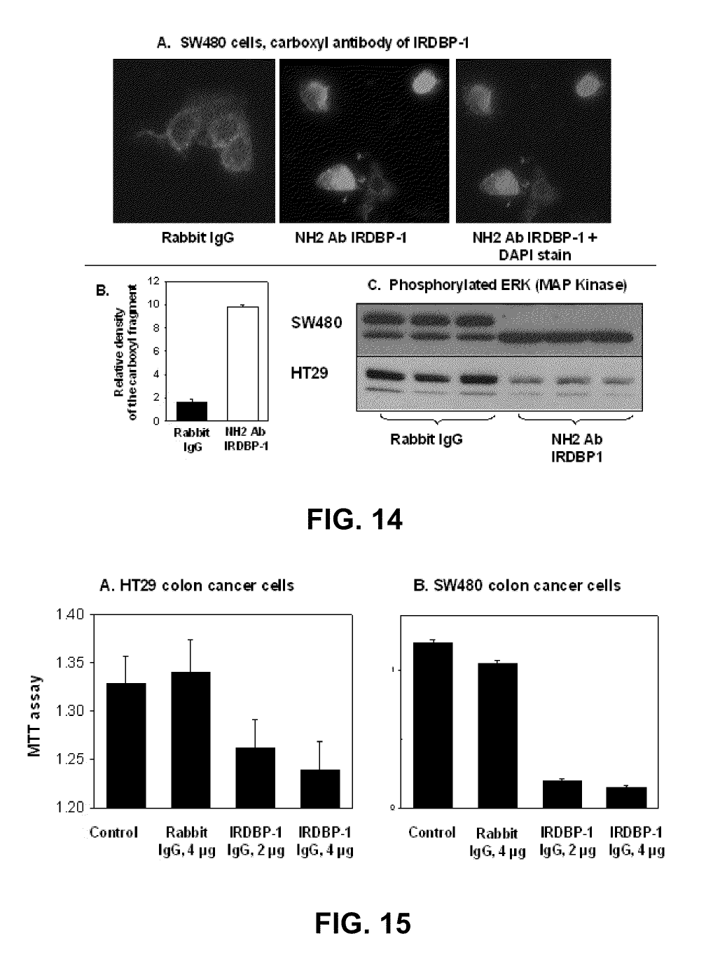

FIG. 14 illustrates the effect of antibody neutralization of IRDBP-1 on nuclear translocation and cell signaling. A. Cultured SW480 cells were preblocked with isotype IgG (rabbit IgG) or NH₂-specific antibody of IRDBP-1 for 8 hrs, then immunostained with the carboxyl-specific antibody of IRDBP-1, without or with DAPI counterstain. B. Cultured SW480 cells were treated with isotype IgG or NH₂-specific antibody of IRDBP-1, and the effect on the expression of the carboxyl fragment of IRDBP-1 was measured by western blotting, and quantitated by densitometer. C. Cultured SW480 and HT29 colon cancer cells were treated with isotype IgG or NH₂-specific antibody of IRDBP-1, and phosphorylation of Erk assessed by western blotting. The epitopes for the NH₂- and COOH-specific antibodies of IRDBP-1 correspond to SEQ ID NOs: 3 and 4, respectively.

The results showed the overnight exposure to the antibody decreased Erk1 phosphorylation by more than 90% in SW480 cells, and decreased Erk activation by 64±6% in HT29 cells (FIG. 14B). Moreover, the antibody induced nuclear translocation of the transcriptionally active carboxyl fragment in SW480 cells (FIG. 14A).

In FIG. 15A-15B, HT29 and SW480 colon cancer cells were grown in 96-well plate, with isotype IgG or the amino fragment specific antibody of IRDBP-1, and MTT assay conducted to determine cell viability after 24 hrs. n=8/group. P<0.0001 for non-treated cells vs. treated cells. The results showed that in cultured SW480 cells, overnight exposure to the antibody decreased cell proliferation by 84 and 87% at 4 and 8 μg/ml concentrations (both p<0.0001 vs. Isotype and untreated controls) (FIG. 15B). It also decreased proliferation of HT29 cells significantly (FIG. 15A). Since the cells were cultured in the presence of 10% serum, IRDBP-1 NH2-segment specific antibody appeared to inhibit serum-stimulated Erk activation, as well as decrease cell proliferation and induce proteolytic release of the transcriptionally-active carboxyl fragment from membrane-bound holoprotein. Since human colon carcinoma is characterized by decreased nuclear localization of IRDBP-1, use of NI-12-segment specific antibody to induce nuclear translocation of the carboxyl fragment of IRDBP-1 and induce transcription of genes that decrease tumor growth may be useful. Somatic RAS mutations are highly prevalent with human cancers that respond poorly to currently available treatments, e.g., carcinomas of the lung, pancreas, colon, and melanomas (Denayer et al. (2008) “Clinical and molecular aspects of RAS related disorders” Journal of Medical Genetics 45: 695-703). RAS has been reported to act upstream of ERK. Therefore, treatment with IRDBP-1 amino segment antibody to decrease ERK activation may also be useful for therapy of other cancer cell types. Therefore, antibody specifically targeted to the IRDBP-1 amino segment may be utilized to decrease cell proliferation by inhibiting cell signaling of growth factors like insulin and by increasing nuclear translocation of the carboxyl fragment of IRDBP-1 to induce cell differentiation.

### Identification of IRDBP-1 Agonists

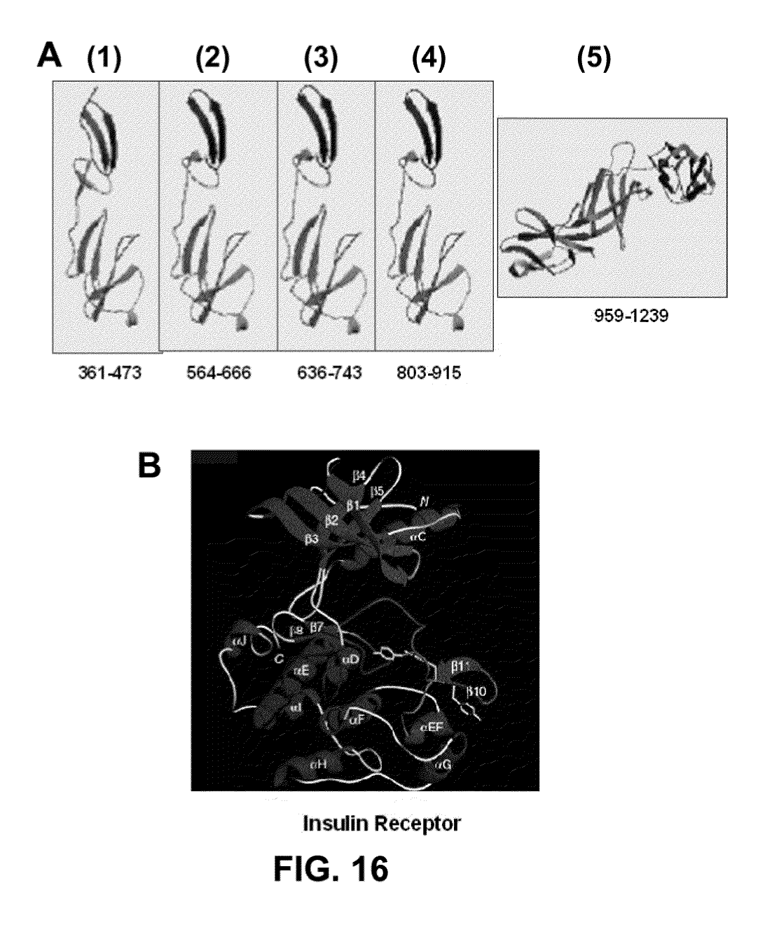

The 3-D structure of IRDBP-1, assessed by homology modelling, was similar to that of insulin receptor (FIG. 16). Since IRDBP-1 was predicted to be a membrane protein and membrane proteins are targets for about 40% of all therapeutic drugs, the structure of the protein may be used to predict the ligand binding sites and the sites for docking small molecules in structure-based drug discovery. As illustrated in FIG. 16, IRDBP-1 forms helix-loop-helix structure, in which 4 parallel 13 strands are linked by loops, and the carboxyl fragment has cup-like structure. The overall structure is very similar to that of insulin receptor (De Meyts (2008) The insulin receptor: a prototype for dimmers, allosteric membrane receptors, Trends in Biochemical Sciences 33: 376-84).

To screen for IRDBP-1 agonists, three tandem repeats of the insulin-response element (IRE) identified in the promoter region of the insulin-like growth factor binding protein-3 (IGFBP-3) gene were inserted into a plasmid that carries the coding region of firefly luciferase to generate a construct comprising IGFBP-3 IRE-linked luciferase reporter gene. Various fragments of IRDBP-1 cDNA were subcloned into a mammalian expression vector (PCR 3.1). The vector comprising the IRDBP-1 cDNA fragment was cotransfected into COS-7 cells with target constructs containing the IGFBP-3 IRE SV40-driven luciferase reporter (pGL3 promoter).

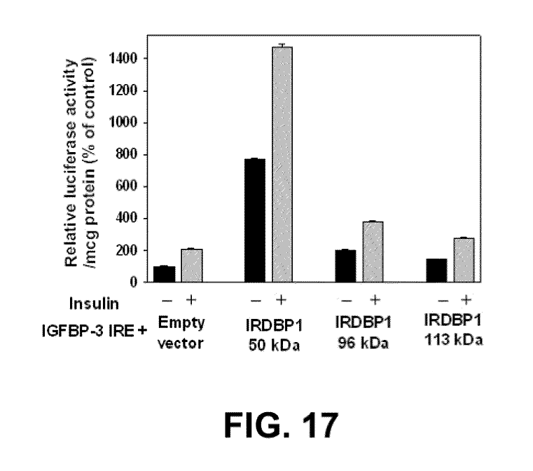

FIG. 17 illustrates a method for screening IRDBP-1 agonists. COS-7 cells were transfected with the 1.4, 2.9, and 3.1 kb of IRDBP-1 cDNA expression vectors and IGFBP-3 IRE SV40 luciferase reporter gene, in the presence (white bars) or absence (black bars) of 10 nM insulin, and the luciferase activity measured.

The results showed that a 1.4 kb IRDBP-1 cDNA (SEQ ID NO: 6) that is translated into the 50 kDa carboxyl fragment of the protein increased the activity of the IRE-linked reporter gene 14-fold but had only a 2-fold effect on the control reporter. The control reporter (without IRE) was the empty pGL3 vector, in which the Luciferease reporter gene was not linked to IGFBP-3 IRE (FIG. 17). The IRDBP-1 2.9 kb and 3.1 kb vectors expressed 96 kDa and 113 kDa proteins, in which the 50 kDa carboxyl fragment is not proteolyzed from the NH2-segment of the protein. Thus, the cDNA constructs having an additional 5′ sequence increased the IRE reporter activity to a much lesser extent than the 50 kDa carboxyl fragment of IRDBP-1. To determine further whether the 50 kDa fragment will be stimulated by an agonist, we added 10⁻⁸ M of insulin to the cells and observed a 3-fold further stimulation of the IRE reporter. Thus, a high throughput screening that utilizes an IRE-driven luciferase reporter in cells expressing the 50 kDa protein fragment of IRDBP-1 could be used to screen for other agonists or antagonists of IRDBP-1. A method of screening other receptor agonists has been reported previously (Guan et al., (2010) “Identification of novel synthetic toll-like receptor 2 agonists by high throughput screening” Journal of Biological Chemistry, 285 (31), 23755-23762).

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method of inhibiting growth of tumor cells, comprising:
introducing a DNA construct encoding IRDBP-1 protein into the tumor cells; and
allowing the DNA construct to express the IRDBP-1 protein in the tumor cells;
thereby inhibiting growth of the tumor cells; wherein the IRDBP-1 protein comprises the amino acid sequence selected from the group consisting of SEQ ID NOs: 2 and 5.

2. The method of claim 1, wherein the tumor cells are present in a mammal.

3. A method of inhibiting growth of tumor cells, comprising:
introducing a DNA construct comprising the nucleotide sequence selected from the group consisting of SEQ ID NOs: 1 and 6; and
allowing the DNA construct to express IRDBP-1 protein in the tumor cells, thereby inhibiting growth of the tumor cells.

4. The method of claim 3, wherein the tumor cells are present in a mammal.

5. A method of inhibiting growth of tumor cells, comprising:
increasing nuclear translocation of the carboxyl fragment of IRDBP-1 in the tumor cells, thereby inhibiting growth of the tumor cells; wherein the carboxyl fragment of IRDBP-1 comprises the amino acid sequence of SEQ ID NO: 5.

6. The method of claim 5, further comprising:
introducing a DNA construct encoding IRDBP-1 protein into the tumor cells; and
allowing the DNA construct to express the IRDBP-1 protein in the tumor cells, thereby inhibiting growth of the tumor cells; wherein the IRDBP-1 protein comprises the amino acid sequence selected from the group consisting of SEQ ID NOs: 2 and 5.

7. The method of claim 5, further comprising:
exposing the tumor cells to an effective amount of an antibody that is specific against one of epitopes chosen from the amino fragment of IRDBP-1, the amino fragment comprising the amino acid sequence of SEQ ID NO: 10.

8. The method of claim 5, wherein the antibody is specific against an epitope comprising
the amino acid sequence of SEQ ID NO: 3.

9. The method of claim 5, wherein the tumor cells are present in a mammal.

10. The method of claim 1, wherein the tumor cells are selected from the group consisting of colon cancer cells, rectal cancer cells, pancreatic cancer cells, breast cancer cells, kidney cancer cells, lymphoma cells, and any combination thereof.

11. An antibody specifically against one of the epitopes chosen from the amino acid sequence selected from the group consisting of SEQ ID NOs: 2 and 10.

12. The antibody of claim 11, specifically against the amino acid sequence of SEQ ID NO: 3.

13. A method of inhibiting growth of tumor cells, comprising:
exposing the tumor cells to an effective amount of the antibody of claim 11, and thereby inhibiting growth of the tumor cells; wherein the antibody is specifically against one of the epitopes chosen from the amino acid sequence of SEQ ID NO: 10.

14. A method of inhibiting growth of tumor cells, comprising:
exposing the tumor cells to an effective amount of the antibody of claim 12, and thereby inhibiting growth of the tumor cells.

15. A method of inhibiting growth of tumor cells in a patient, comprising:
exposing the tumor cells in the patient to an effective amount of the antibody of claim 11, and thereby inhibiting growth of tumor cells in the patient; wherein the antibody is specifically against one of the epitopes chosen from the amino acid sequence of SEQ ID NO: 10.

16. A method of inhibiting growth of tumor cells in a patient, comprising:
exposing the tumor cells in the patient to an effective amount of the antibody of claim 12, and thereby inhibiting growth of tumor cells in the patient.

17. A method for identifying a compound that affects activity of IRDBP-1 in a cell, comprising:
providing a cell transfected with a first vector and a second vector, the first vector comprising a reporter gene operably linked to IGFBP-3 promoter insulin responsive element (IRE) and the second vector comprising a DNA insert encoding a polypeptide comprising the amino acid sequence of SEQ ID NO: 5 operably linked to a constitutive promoter;
exposing the cell to a test compound;
detecting the level of the expression product of the reporter gene expression in the cell exposed to the test compound; and
comparing the level of the expression product of the reporter gene expression in the cell exposed to the test compound with the level of the expression product of the reporter gene expression in the cell not exposed to the test compound to detect whether the test compound affects activity of IRDBP-1 in the cell;
wherein an increase in the level of the expression product of the reporter gene expression in the presence of the test compound relative to the level of the expression product of the reporter gene expression in the absence of the test compound is an indication of a potential agonist of the test compound.

18. The method of claim 17, wherein the reporter gene is one chosen from firefly luciferase and β-galactosidase, and green fluorescent protein.

19. The method of claim 17, wherein the constitutive promoter is a CMV promoter.

20. A method of identifying a compound that affects the activity of IRDBP-1 in a cell comprising:
providing a cell transfected with a nucleic acid insert comprising:
(a) a constitutive promoter;
(b) a cDNA encoding IRDBP-1, the cDNA operably linked to the constitutive promoter and the IRDBP-1 comprising the amino acid sequence of SEQ ID NO: 2; and
(c) a reporter gene operably linked to the cDNA at the 3′ end thereof;
exposing the cell to a test compound;
detecting the expression product of reporter gene expression in the cell exposed to the test compound; and
comparing the intracellular localization of the expression product of the reporter gene expression in the cell exposed to the test compound with the intracellular localization of the expression product of the reporter gene expression in the cell not exposed to the test compound to determine whether the test compound affects the activity of IRDBP-1 in the cell;
wherein an increase in nuclear detection of the expression product of the reporter gene expression is an indication of a potential agonist of the test compound.


SEQUENCE LISTING (10 sequences)

SEQ ID NO: 1 - DNA, 6331 bp, Homo sapiens
gcctcgtccataatgatggctgcatggccacttccggcccaagagggatccaagaggcagcgcggtccgatcggagagcacccttaccgtgggcacctgcacgagcggcccaggctgtcaaatcagtgaccaggatttgtttgcaacaccagcgggccccaaatatgagggtgttagtgtctcaggctgtggtcttccaagtggcagaccagaccgcagactggggtagcaggctttttctgtagagggccaggtagtgactattttcagcttttcaggccacagggtctctgctgttgtcacaaaaaagcagccgtagacagtaagtacgtgagtgtgctcagccgtgctcccacaaaccctgtttctgaaaccaggctggggtccacagaggatggatggccacatctgacccaaggcctgtttgtgtatgacctgtgaactaagaatgttcttaatacttttaaagggctgcaaaaacaaaaagaacaaaaaagaatatgcaacagagaccacgtgtggtccacagggccagaaatatttgcgatctggcccttcccagaaagtctgctggcccttgggcagaccgcaccatttgcaacaaccaaaattcagtaaaacttcaatgtttctggtccttaagagtcctaaggccagggtggcagcttccagcccagccccaatactcactggtcttctgctgtctcccagagttggcgctggggctggacagccactgcccaggggacgccattgggctccagcttagccggaccatccttgaaccaggcccaggcatcgggggccccgtgactcgagcaccccgcagagccagtggctcacgtcaggcactggaccggaaggtgaacaacaacgggatcatctccttcctgaaggaggtttctcagttcaccccagtggccttccccattgccaaggaccgctgcgtggtggcagccttctgggcagatgtgaacaaccggcgtgcaggcgacgtgtactaccgggaggccaccgacccagccatgctgcgccgagccacggaggacgtcaggcactacttccccgagctcctggacttcaatgccacctgggtttttgttgccacctggtaccgagtgaccttctttggaggcagttcctcatcccctgtcaacacattccagactgtgctcatcacagacggcaagctctccttcaccatcttcaactatgagtccatcgtgtggaccacaggcacacacgccagcagcgggggcaacgccactggcctcgggggcatcgcagcccaggctggcttcaacgcaggcgatgggcagcgttacttcagtatccccggctcgcgcacagcagacatggccgaggtggagaccaccaccaacgtgggtgtgcccgggcgctgggcgttcagaatcgatgatgcccaggtgcgcgtggggggctgcggccatacaacgtccgtgtgcctggccctgcgcccctgcctcaacggcggcaagtgcatcgacgactgcgtcacgggcaacccctcctacacctgctcctgcctctcgggcttcacggggcggaggtgccacctggacgtgaacgaatgtgcctcccagccctgtcagaatggtgggacctgtactcacggcatcaacagtttccgctgccagtgcccggctggctttgggggacccacctgtgagacagcccaatccccctgtgacaccaaagagtgtcaacatggtggccagtgccaggtggagaatggctctgcggtgtgtgtgtgccaggccggatacaccggagcagcctgcgagatggatgtggacgactgcagccctgacccctgcctgaatggaggctcttgtgttgacctagtggggaattacacctgcttgtgtgccgagcccttcaagggacttcgctgtgagacaggagaccatccagtgccagacgcctgcctctcggccccttgccacaatgggggcacctgtgtggatgcggaccagggctacgtgtgcgagtgccccgaaggcttcatgggcctggactgcagggagagagtccccgatgactgtgagtgccgcaacggaggcagatgcctgggcgccaacaccaccctctgccagtgccccctgggattctttgggcttctctgtgaatttgaaatcacagccatgccctgcaacatgaacacacagtgcccagatgggggctactgcatggagcacggcgggagctacctctgcgtctgccacaccgaccacaatgccagccactccctgccatcaccctgcgactcggacccctgcttcaacggaggctcctgcgatgcccatgacgactcctacacctgcgagtgcccgcgcgggttccacggcaagcactgcgagaaagcccggccacacctgtgcagctcagggccctgccggaacgggggcacgtgcaaggaggcgggcggcgagtaccactgcagctgcccctaccgcttcactgggaggcactgtgagatcgggaagccagactcgtgtgcctctggcccctgtcacaacggcggcacctgcttccactacattggcaaatacaagtgtgactgtcccccaggcttctccgggcggcactgcgagatagccccctccccctgcttccggagcccgtgtgtgaatgggggcacctgcgaggaccgggacacggatttcttctgccactgccaagcagggtacatgggacgccggtgccaggcagaggtggactgcggccccccggaggaggtgaagcacgccacactgcgcttcaacggcacgcggctgggcgcggtggccctgtatgcatgtgaccgtggctacagcctgagcgcccccagccgcatccgggtctgccagccacacggtgtctggagtgagcctccccagtgccttgaaatcgatgagtgccggtctcagccgtgcctgcatgggggctcttgtcaggaccgcgttgctgggtacctgtgcctctgcagcacaggctatgagggcgcccactgtgagctggagagggatgagtgccgagctcacccgtgcagaaatggagggtcctgcaggaacctcccaggggcctatgtctgccggtgccctgcaggcttcgttggagtccactgtgagacagaggtggacgcctgcgactccagcccctgccagcatggaggccggtgtgagagcggcggcggggcctacctgtgcgtctgcccagagagcttcttcggctaccactgcgagacagtgagtgacccctgcttctccagcccctgtgggggccgtggctattgcctggccagcaacggctcccacagctgcacctgcaaagtgggctacacgggcgaggactgcgccaaagagctcttcccaccgacggccctcaagatggagagagtggaggagagtggggtctctatctcctggaacccgcccaatggtccagccgccaggcagatgcttgatggctacgcggtcacctacgtctcctccgacggctcctaccgccgcacagactttgtggacaggacccgctcctcgcaccagctccaggccctggcggccggcagggcctacaacatctccgtcttctcagtgaagcgaaacagtaacaacaagaatgacatcagcaggcctgccgtgctgctggcccgcacgcgaccccgccctgtggaaggcttcgaggtcaccaatgtgacggctagcaccatctcagtgcagtgggccctgcacaggatccgccatgccaccgtcagtggggtccgtgtgtccatccgccaccctgaggccctcagggaccaggccaccgatgtggacaggagtgtggacaggttcacctttagggccctgctgcctgggaagaggtacaccatccagctgaccaccctcagtgggctcaggggagaggagcaccccacagagagcctggccaccgcgccgacgcacgtgtggacccggcccctgcctccagcaaacctgaccgccgcccgagtcactgccacctctgcccacgtggtctgggatgccccgactccaggcagcttgctggaggcttatgtcatcaatgtgaccaccagccagagcaccaagagccgctatgtccccaacgggaagctggcgtcctacacggtgcgcgacctgctgccgggacggcggtaccagctctctgtgatagcagtgcagagcacggagctcgggccgcagcacagcgagcccgcccacctctacatcatcacctcccccagggatggcgctgacagacgctggcaccagggaggacaccaccctcgggtgctcaagaacagaccgcccccggcgcgcctgccggagctgcgcctgctcaatgaccacagcgcccccgagacccccacccagccccccaggttctcggagcttgtggacggcagaggaagagtgagcgccaggttcggtggctcacccagcaaagcagccaccgtgagatcacaacccacagcctcggcgcagctcgagaacatggaggaagcccccaagcgggtcagcctggccctccagctccctgaacacggcagcaaggacatcggaaacgtccctggcaactgttcagaaaacccctgtcagaacggaggcacttgtgtgccgggcgcagacgcccacagctgtgactgcgggccagggttcaaaggcagacgctgcgagctcgcctgtataaaggtgtcccgcccctgcacaaggctgttctccgagacaaaggcctttccagtctgggagggaggcgtctgtcaccacgtgtataaaagagtctaccgagttcaccaagacatctgcttcaaagagagctgtgaaagcacaagcctcaagaagaccccaaacaggaaacaaagtaagagtcagacactggagaaatcttaaggatttaagacgttcttgttacactccaccaacctcacgagtttctaacacccaggaagatgaggtctaaaaactggatgaaaaaggacaccctgagaaaaggtcctagctggagtcagtcccctctgtgacctctctcctcaggcctctagaggacagatggccaggcctgtgcacacaccagcccaccctgagagacccctctgggaccaaccacctgtgagtcctgcgatgcgtttaagcagcctgtgccctcacccaagctgcagttcctgaaggtgtagtctgtgtctctgcggatgagatgacagctcgccattccccggaatcagtgaggctgtcagtcagccacgcttctgcagtatgcagaaacctgttcttagactccaaagccagagaaagaattctcccttcgaggcccaacaaattgagaaggaactgtgatggaccacttccaaaacagagacgggggcaggggctgaagggcagagaccaggtgatgtcagaaggaaagccgggttgcagacacagccgcccctgctctggtcctccagcgtgtttatgacgctcgtgcaggtcgacgagccatcctatggactagttaacactaaggtggagttcagacttttttagacaacggcgcgactggcagcctttctctatcaagggtcagacggtaaacgttttcagctttgcagaccagaggtccctgtggctacagtagcgcagacacagccacaggcatgtcattgaatggctgcggctatgttccaataaaaacttatttacaataacaggtggtggccaaattggcccatgggccttatttggtgaaccctgttctatgagatcacctaggcttcagccttaaacagtggaagccatcccctgaatgacaagtcacaagggtatcaaagaaagacccctgaattttcatggaaaaagctattcagacccctgcttggaaagctaaggcacactgccacgaagcagcaaggacgccttacaagtctcagtgcaacagagatggacacctgggctgggctggacaatgtttaaggttccttttagtccatgactcaagtgatactgttttaggctatcaggtagtaaacacgatcttagacatccccatctttgtaagcagaacagtacggcacttcaccacatctgcttcccaccatgcttctaagcagctgtcttccccctgctaatgttacaaccaaagcagccaccccacctcctctcgtgttgagcctcacgaccgctgacccagctggaaagccagcgccctgccgcgtcaccctgactctgctcagagccagcattccagccacaaagagggcctccttcctttcctctttcataaaaatgttttttgaagagttagagtatattttaggctttttatctttattaaaatttcatgtgcatgtgta

SEQ ID NO: 2 - protein, 1463 aa, Homo sapiens
MQQRPRVVHRARNICDLALPRKSAGPWADRTICNNQNSVKLQCFWSLRVLRPGWQLPAQPQYSLVFCCLPELALGLDSHCPGDAIGLQLSRTILEPGPGIGGPVTRAPRRASGSRQALDRKVNNNGIISFLKEVSQFTPVAFPIAKDRCVVAAFWADVNNRRAGDVYYREATDPAMLRRATEDVRHYFPELLDFNATWVFVATWYRVTFFGGSSSSPVNTFQTVLITDGKLSFTIFNYESIVWTTGTHASSGGNATGLGGIAAQAGFNAGDGQRYFSIPGSRTADMAEVETTTNVGVPGRWAFRIDDAQVRVGGCGHTTSVCLALRPCLNGGKCIDDCVTGNPSYTCSCLSGFTGRRCHLDVNECASQPCQNGGTCTHGINSFRCQCPAGFGGPTCETAQSPCDTKECQHGGQCQVENGSAVCVCQAGYTGAACEMDVDDCSPDPCLNGGSCVDLVGNYTCLCAEPFKGLRCETGDHPVPDACLSAPCHNGGTCVDADQGYVCECPEGFMGLDCRERVPDDCECRNGGRCLGANTTLCQCPLGFFGLLCEFEITAMPCNMNTQCPDGGYCMEHGGSYLCVCHTDHNASHSLPSPCDSDPCFNGGSCDAHDDSYTCECPRGFHGKHCEKARPHLCSSGPCRNGGTCKEAGGEYHCSCPYRFTGRHCEIGKPDSCASGPCHNGGTCFHYIGKYKCDCPPGFSGRHCEIAPSPCFRSPCVNGGTCEDRDTDFFCHCQAGYMGRRCQAEVDCGPPEEVKHATLRFNGTRLGAVALYACDRGYSLSAPSRIRVCQPHGVWSEPPQCLEIDECRSQPCLHGGSCQDRVAGYLCLCSTGYEGAHCELERDECRAHPCRNGGSCRNLPGAYVCRCPAGFVGVHCETEVDACDSSPCQHGGRCESGGGAYLCVCPESFFGYHCETVSDPCFSSPCGGRGYCLASNGSHSCTCKVGYTGEDCAKELFPPTALKMERVEESGVSISWNPPNGPAARQMLDGYAVTYVSSDGSYRRTDFVDRTRSSHQLQALAAGRAYNISVFSVKRNSNNKNDISRPAVLLARTRPRPVEGFEVTNVTASTISVQWALHRIRHATVSGVRVSIRHPEALRDQATDVDRSVDRFTFRALLPGKRYTIQLTTLSGLRGEEHPTESLATAPTHVWTRPLPPANLTAARVTATSAHVVWDAPTPGSLLEAYVINVTTSQSTKSRYVPNGKLASYTVRDLLPGRRYQLSVIAVQSTELGPQHSEPAHLYIITSPRDGADRRWHQGGHHPRVLKNRPPPARLPELRLLNDHSAPETPTQPPRFSELVDGRGRVSARFGGSPSKAATVRSQPTASAQLENMEEAPKRVSLALQLPEHGSKDIGNVPGNCSENPCQNGGTCVPGADAHSCDCGPGFKGRRCELACIKVSRPCTRLFSETKAFPVWEGGVCHHVYKRVYRVHQDICFKESCESTSLKKTPNRKQSKSQTLEKS

SEQ ID NO: 3 - protein, 17 aa, Artificial Sequence
  Amino terminus epitope of IRDBP-1 protein
TRAPRRASGSRQALDRK

SEQ ID NO: 4 - protein, 18 aa, Artificial Sequence
  Carboxyl terminus epitope of IRDBP-1 protein
SPRDGADRRWHQGGHHPR

SEQ ID NO: 5 - protein, 460 aa, Homo sapiens
RTDFVDRTRSSHQLQALAAGRAYNISVFSVKRNSNNKNDISRPAVLLARTRPRPVEGFEVTNVTASTISVQWALHRIRHATVSGVRVSIRHPEALRDQATDVDRSVDRFTFRALLPGKRYTIQLTTLSGLRGEEHPTESLATAPTHVWTRPLPPANLTAARVTATSAHVVWDAPTPGSLLEAYVINVTTSQSTKSRYVPNGKLASYTVRDLLPGRRYQLSVIAVQSTELGPQHSEPAHLYIITSPRDGADRRWHQGGHHPRVLKNRPPPARLPELRLLNDHSAPETPTQPPRFSELVDGRGRVSARFGGSPSKAATVRSQPTASAQLENMEEAPKRVSLALQLPEHGSKDIGNVPGNCSENPCQNGGTCVPGADAHSCDCGPGFKGRRCELACIKVSRPCTRLFSETKAFPVWEGGVCHHVYKRVYRVHQDICFKESCESTSLKKTPNRKQSKSQTLEKS

SEQ ID NO: 6 - DNA, 1380 bp, Homo sapiens
cgcacagactttgtggacaggacccgctcctcgcaccagctccaggccctggcggccggcagggcctacaacatctccgtcttctcagtgaagcgaaacagtaacaacaagaatgacatcagcaggcctgccgtgctgctggcccgcacgcgaccccgccctgtggaaggcttcgaggtcaccaatgtgacggctagcaccatctcagtgcagtgggccctgcacaggatccgccatgccaccgtcagtggggtccgtgtgtccatccgccaccctgaggccctcagggaccaggccaccgatgtggacaggagtgtggacaggttcacctttagggccctgctgcctgggaagaggtacaccatccagctgaccaccctcagtgggctcaggggagaggagcaccccacagagagcctggccaccgcgccgacgcacgtgtggacccggcccctgcctccagcaaacctgaccgccgcccgagtcactgccacctctgcccacgtggtctgggatgccccgactccaggcagcttgctggaggcttatgtcatcaatgtgaccaccagccagagcaccaagagccgctatgtccccaacgggaagctggcgtcctacacggtgcgcgacctgctgccgggacggcggtaccagctctctgtgatagcagtgcagagcacggagctcgggccgcagcacagcgagcccgcccacctctacatcatcacctcccccagggatggcgctgacagacgctggcaccagggaggacaccaccctcgggtgctcaagaacagaccgcccccggcgcgcctgccggagctgcgcctgctcaatgaccacagcgcccccgagacccccacccagccccccaggttctcggagcttgtggacggcagaggaagagtgagcgccaggttcggtggctcacccagcaaagcagccaccgtgagatcacaacccacagcctcggcgcagctcgagaacatggaggaagcccccaagcgggtcagcctggccctccagctccctgaacacggcagcaaggacatcggaaacgtccctggcaactgttcagaaaacccctgtcagaacggaggcacttgtgtgccgggcgcagacgcccacagctgtgactgcgggccagggttcaaaggcagacgctgcgagctcgcctgtataaaggtgtcccgcccctgcacaaggctgttctccgagacaaaggcctttccagtctgggagggaggcgtctgtcaccacgtgtataaaagagtctaccgagttcaccaagacatctgcttcaaagagagctgtgaaagcacaagcctcaagaagaccccaaacaggaaacaaagtaagagtcagacactggagaaatct

SEQ ID NO: 7 - DNA, 29 bp, Artificial Sequence
  siRNA sense
aagcatggctgtgatttcaaacctgtctc

SEQ ID NO: 8 - DNA, 29 bp, Artificial Sequence
  siRNA antisense
aatttgaaatcacagccatgccctgtctc

SEQ ID NO: 9 - DNA, 34 bp, Artificial Sequence
  IGFBP-3 IRE primer
aattcaagggtatccaggaaagtctccttctaag

SEQ ID NO: 10 - protein, 530 aa, Homo sapiens
MQQRPRVVHRARNICDLALPRKSAGPWADRTICNNQNSVKLQCFWSLRVLRPGWQLPAQPQYSLVFCCLPELALGLDSHCPGDAIGLQLSRTILEPGPGIGGPVTRAPRRASGSRQALDRKVNNNGIISFLKEVSQFTPVAFPIAKDRCVVAAFWADVNNRRAGDVYYREATDPAMLRRATEDVRHYFPELLDFNATWVFVATWYRVTFFGGSSSSPVNTFQTVLITDGKLSFTIFNYESIVWTTGTHASSGGNATGLGGIAAQAGFNAGDGQRYFSIPGSRTADMAEVETTTNVGVPGRWAFRIDDAQVRVGGCGHTTSVCLALRPCLNGGKCIDDCVTGNPSYTCSCLSGFTGRRCHLDVNECASQPCQNGGTCTHGINSFRCQCPAGFGGPTCETAQSPCDTKECQHGGQCQVENGSAVCVCQAGYTGAACEMDVDDCSPDPCLNGGSCVDLVGNYTCLCAEPFKGLRCETGDHPVPDACLSAPCHNGGTCVDADQGYVCECPEGFMGLDCRERVPDDCECRNGGRC